(19) United States
(12) Patent Application Publication
(10) Pub. No.: US 2021/0163468 A1
(43) Pub. Date: Jun. 3, 2021

(54) PEROXISOME PROLIFERATOR-ACTIVATED RECEPTOR GAMMA SELECTIVE AGONISTS FOR INHIBITION OF RETINAL PIGMENT EPITHELIUM DEGENERATION OR GEOGRAPHIC ATROPHY

(71) Applicant: The Schepens Eye Research Institute, Inc., Boston, MA (US)

(72) Inventors: Yin Shan Eric Ng, North Billerica, MA (US); Gopalan Gnanaguru, Cambridge, MA (US); Ashley Mackey, Somerville, MA (US); Patricia A. D'Amore, Newton, MA (US)

(21) Appl. No.: 17/171,528

(22) Filed: Feb. 9, 2021

Related U.S. Application Data
(62) Division of application No. 16/091,439, filed on Oct. 4, 2018.
(60) Provisional application No. 62/318,165, filed on Apr. 4, 2016.

(51) Int. Cl. C07D 417/12; A61K 31/517; A61K 9/08; A61K 9/00; A61K 31/506; A61K 31/427

(52) U.S. Cl. CPC C07D 417/12; A61K 31/517; A61K 9/08; A61K 31/427; A61K 9/0048; A61K 31/506; A61K 9/0019

(57) ABSTRACT

The invention provides a solution to the clinical problem of retinal pigment epithelium (RPE) degeneration or geographic atrophy (GA) associated AMD. PPARγ selective agonists, e.g., troglitazone and analogs thereof are used to reduce or inhibit RPE degeneration, GA, and/or the progression of dry AMD.

### CROSS-REFERENCES TO RELATED APPLICATIONS

This application claims the benefit of U.S. Provisional Application No. 62/318,165, filed Apr. 4, 2016, the contents of which is incorporated herein in its entirety and for all purposes.

### BACKGROUND OF THE INVENTION

Age-related macular degeneration (AMD) is a disease associated with aging that progressively destroys a person's sharp, central vision. It is generally thought to progress along a continuum from atrophic or “dry” AMD to either advanced dry AMD with geographic atrophy (GA) of the retinal pigment epithelium or neovascular “wet” AMD with choroidal neovascularization. Dry AMD, the early form of AMD, accounts for 85 percent to 90 percent of all cases. It is characterized by the presence of fatty deposits called drusen in the macula. The collection of small, round, yellow-white drusen is a key identifier for AMD. Approximately 15 million people in the United States have AMD, and more than 1.7 million Americans have the advanced form of the disease. Due to the aging baby boomer population, the National Eye Institute estimates that the prevalence of advanced AMD will grow to nearly 3 million by 2020. It afflicts an estimated 30 million to 50 million people worldwide and is the leading cause of severe vision loss in Western societies. There is currently no treatment available to treat dry macular degeneration at any stage including GA.

### SUMMARY OF THE INVENTION

Provided herein is a solution to the clinical problem of retinal pigment epithelium (RPE) degeneration or GA associated with AMD, including dry AMD. In various embodiments, PPARγ-selective agonists, e.g., troglitazone, are used to reduce or inhibit RPE degeneration and GA. For example, methods of treating dry AMD comprising administering a PPAR agonist to a subject are provided herein. Non-limiting examples of PPAR agonists, include troglitazone and anologues thereof.

Accordingly, the invention features a method of reducing retinal pigment epithelium (RPE) cell death, comprising contacting the RPE cells with a PPARγ-selective agonist. Preferably, the agonist does not comprise substantial PPARα, β, or δ agonist activity. In one example, the agonist comprises a thiazolidinedione (TZD) compound such as troglitazone or an analogue thereof. In another example, the agonist may be a compound comprising a TZD domain or a derivative thereof that may activate PPARs, for example, by having specificity for PPARγ (gamma). A non-limiting example of the agonist includes troglitazone, i.e. (RS)-5-(4-[(6-hydroxy-2,5,7,8-tetramethylchroman-2-yl)methoxy]benzyl)thiazolidine-2,4-dione, having the following structure:

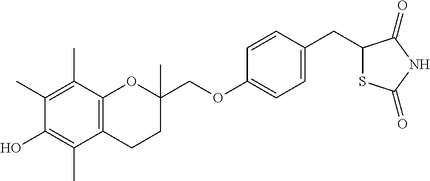 [SMILES: Cc1c(C)c2c(c(C)c1O)CCC(C)(COc1ccc(CC3SC(=O)NC3=O)cc1)O2]

In some embodiments, the agonist is administered to a subject diagnosed with or suffering from or at risk of developing GA. In certain embodiments, a subject is a human. In various embodiments, a subject is at least 40 years old. In some embodiments, a subject is at least 50 years old. In certain embodiments, the subject has been diagnosed with dry AMD. In some embodiments, the subject. has been diagnosed with wet AMD. In various embodiments, the subject has not been diagnosed with wet AMD.

The agonist is administered locally to the eye, e.g., by ocular injection such as intravitreal injection, by topical administration such as eye drop, by periorbital injection such as subtenon injection or is systemically, e.g., orally, delivered.

In various implementations of the present subject matter, a method of reducing the size of GA or inhibiting the progression of GA and/or AMD (including dry AMD, e.g., advanced dry AMD) is carried out by administering to a subject a PPARγ-selective agonist. In certain embodiments, the agonist is administered locally to the eye or is administered systemically. In various embodiments, a dose is administered to achieve an ocular agonist concentration of about 35, 30, 25, 20, 15, 10, or 5 μM, or less than about 35, 30, 25, 20, 15, 10, or 5 μM, based on the, measurement, assumption, and/or estimation that the volume of the eye is about 4, 5, 6, 7, 8, 9, 10, 12, 14, 16, 18, 20, or 4-20 mL. In embodiments, the agonist is administered at an oral dose of less than about 400, 350, 250, 200, 150, 100, 50, 25, 20, 10, or 5 mg, or about 5-400, 5-100, 5-50, or 5-10 mg. In some embodiments, the agonist is administered at an oral dose of less than about 400, 350, 250, 200, 150, 100, 50, 25, 20, 10, or 5 mg QD, or about 5-400, 5-100, 5-50, or 5-10 mg QD. In certain embodiments, a dose of about 0.1, 0.5, 1, 2, 3, 4, 5, 5, 6, 7, 8, 9, 10, 11, 12, 13,14, 15, 16, 17, 18, 19, 20, 21, 22, 23, 24, 25, 30, 35, 40, 45, 50, 55, 60, 65, 70, 75, 80, 85, 90, 95, 100, 125, 150, 200, 0.1-5, 0.1-10, 0.1-20, 0.5-5, 0.5-10, 5-10, 5-100, 5-25, 5-50, 25-50, 25-75, 25-100, 50-75, 50-100, 50-200, 75-150, 75-200, 100-150, 100-200, or 100-200 μg is administered to the subject. In various embodiments, a dose of about 0.1, 0.5, 1, 2, 3, 4, 5, 5, 6, 7, 8, 9, 10, 11, 12, 13, 14, 15, 16, 17, 18, 19, 20. 21, 22, 23, 24, 25, 30, 35, 40, 45, 50, 55, 60, 65, 70, 75, 80, 85, 90, 95, 100, 125, 150, 200, 0.1-5, 0.1-10, 0.1-20, 0.5-5, 0.5-10, 5-10, 5-100, 5-25, 5-50, 25-50, 25-75, 25-100, 50-75, 50-100, 50-200, 75-150, 75-200, 100-150, 100-200, or 100-200 μg or less than about 0.1, 0.5, 1, 2, 3, 4, 5, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 125, 150, or 200 μg is administered (e.g., topically or by injection) into or onto one or both eyes of a subject. In certain embodiments, a dose is administered at least about 1, 2, 3, 4, 5, 6, 7, 8, 9, 10, 11, 12 or more times per day, week, month, or year.

In various embodiments, an agonist (such as troglitazone) is administered systemically. For example, the agonist is administered systemically at a dose that is below liver toxicity dose. Doses for systemic administration are generally at least or about a thousand (or thousands) fold lower than oral doses. In some embodiments, troglitazone is administered at a dose that is less than about 400 mg/day (e.g., less than about 350, 250, 200, 150, 100, 50, 25, 20, 10, or 5 mg/day). In certain embodiments relating to ocular injection, the systemic levels of the drug are very low (e.g., thousands fold lower than oral dosing) and a subject does not show clinical signs (e.g. by blood test) of liver dysfunction or toxicity.

In some embodiments, the agonist is administered before detection of drusen in the eye. In certain embodiments, the agonist is administered prior to the development of (i.e., in a subject who has not developed) dry AMD, advanced dry AMD, and/or GA in the eye. In various embodiments, the agonist is administered to a subject who has not developed wet AMD. In some embodiments, the agonist is administered to a subject who has not developed advanced wet AMD.

Also provided herein is a method of reducing the size of GA or inhibiting the progression of GA, comprising administering to a subject a retinoid X receptor (RXR) antagonist.

The present subject matter also encompasses a troglitazone analogue for reducing RPE cell death, reducing the size of GA, and/or inhibiting progression of GA, AMD, and/or advanced AMD.

Aspects of the present subject matter provide a method for reducing RPE cell death, reducing the size of GA, inhibiting progression of GA, and/or inhibiting the progression of dry AMD (including, e.g., advanced dry AMD), comprising contacting a RPE cell with a compound, wherein the compound has a structure according to Formula (I),

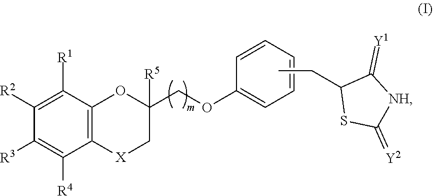

or a pharmaceutically acceptable salt thereof, wherein

each of R¹ and R² is independently hydrogen, C₁-C₆ alkyl, or a C₁-C₆ alkoxy group, or R¹ and R² combine to form a —O(CH₂)ₙO— group;

R³ is hydrogen, —R^{3A}, —OH, —OR^{3A}, —OC(═O)R^{3A}, or —OC(═O)OR^{3A};

R^{3A} is C₁-C₆ alkyl, C₆-C₁₀ aryl, 5- to 6-membered heteroaryl, C₃-C₈ cycloalkyl, or a three- to eight-membered heterocycloalkyl;

R⁴ is a hydrogen atom or a C₁-C₆ alkyl group;

R⁵ is a hydrogen atom or a C₁-C₆ alkyl group;

X is —CH₂—, —C(═O)—, or —CHOR^{X}—;

R^{X} is C₁-C₆ alkyl, C₆-C₁₀ aryl, 5- to 6-membered heteroaryl, C₃-C₈ cycloalkyl, or a three- to eight-membered heterocycloalkyl;

each of Y¹ and Y² is independently O, S, or NH;

m is 1, 2, or 3; and

n is 1, 2, 3, or 4.

In a non-limiting example, the compound described above has a structure according to Formula (II),

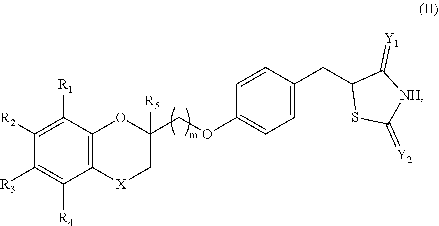

or a pharmaceutically acceptable salt thereof.

Aspects of the present subject matter also provide a method for reducing RPE cell death, reducing the size of GA, inhibiting progression of GA, and/or inhibiting the progression of dry AMD (including, e.g., advanced dry AMD), comprising contacting a RPE cell with a compound, wherein the compound has a structure according to Formula (III),

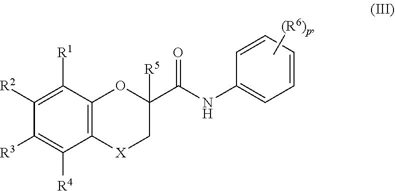

or a pharmaceutically acceptable salt thereof, wherein:

each of R¹ and R² is independently hydrogen, C₁-C₆ alkyl, or a C₁-C₆ alkoxy group, or R¹ and R² combine to form a —O(CH₂)ₙO— group;

R³ is hydrogen, —R^{3A}, —OH, —OR^{3A}, —OC(═O)R^{3A}, or —OC(═O)OR^{3A};

R^{3A} is C₁-C₆ alkyl, C₆-C₁₀ aryl, 5- to 6-membered heteroaryl, C₃-C₈ cycloalkyl, or a three- to eight-membered heterocycloalkyl;

R⁴ is a hydrogen atom or a C₁-C₆ alkyl group;

R⁵ is a hydrogen atom or a C₁-C₆ alkyl group;

R⁶, when present, is independently selected from halogen, —CN, —NO₂, C₁-C₆ alkyl, C₆-C₁₀ aryl, 5- to 6-membered heteroaryl, cycloalkyl, and a three- to eight-membered heterocycloalkyl;

X is —CH₂—, —C(═O)—, or —CHOR^{X}—;

each R^{X} is independently C₁-C₆ alkyl, C₆-C₁₀ aryl, 5- to 6-membered heteroaryl, C₃-C₈ cycloalkyl, or a three- to eight-membered heterocycloalkyl;

each of Y¹ and Y² is independently O, S, or NH;

n is 1, 2, 3, or 4; and

p is 0, 1, 2, 3, 4, or 5.

In various embodiments, in any one of the structures described above, wherein X can be —CH₂—.

Also included is a method for reducing RPE cell death, reducing the size of GA, inhibiting progression of GA, and/or inhibiting the progression of dry AMD (including, e.g., advanced dry AMD), comprising contacting a RPE cell with a compound, wherein the compound has a structure according to Formula (IV),

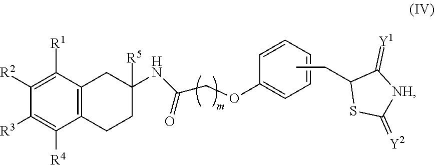

or a pharmaceutically acceptable salt thereof, wherein:

each of R¹ and R² is independently hydrogen, C₁-C₆ alkyl, or a C₁-C₆ alkoxy group, or R¹ and R² combine to form a —O(CH₂)ₙO— group;

R³ is hydrogen, —R^{3A}, —OH, —OR^{3A}, —OC(═O)R^{3A}, or —OC(═O)OR^{3A};

R^{3A} is C₁-C₆ alkyl, C₆-C₁₀ aryl, 5- to 6-membered heteroaryl, C₃-C₈ cycloalkyl, or a three- to eight-membered heterocycloalkyl;

R⁴ is a hydrogen atom or a C₁-C₆ alkyl group;

R⁵ is a hydrogen atom or a C₁-C₆ alkyl group;

each of Y¹ and Y² is independently O, S, or NH;

m is 1, 2, or 3; and

n is 1, 2, 3, or 4.

For example, the compound may have a structure according to Formula (V),

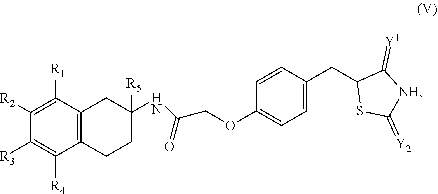

or a pharmaceutically acceptable salt thereof.

In some embodiments, in any one of the structures described above, R⁵ may be hydrogen or unsubstituted C₁-C₆ alkyl.

Also provided herein is a method for reducing RPE cell death, reducing the size of GA, inhibiting progression of GA, and/or inhibiting the progression of dry AMD (including, e.g., advanced dry AMD), comprising contacting a RPE cell with a compound, wherein the compound has a structure according to Formula (VI),

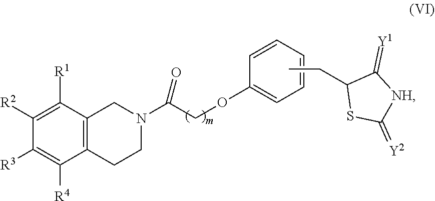

or a pharmaceutically acceptable salt thereof, wherein:

each of R¹ and R² is independently hydrogen, C₁-C₆ alkyl, or a C₁-C₆ alkoxy group, or R¹ and R² combine to form a —O(CH₂)ₙO— group;

R³ is hydrogen, —R^{3A}, —OH, —OR^{3A}, —OC(═O)R^{3A}, or —OC(═O)OR^{3A};

R^{3A} is C₁-C₆ alkyl, C₆-C₁₀ aryl, 5- to 6-membered heteroaryl, C₃-C₈ cycloalkyl, or a three- to eight-membered heterocycloalkyl;

R⁴ is a hydrogen atom or a C₁-C₆ alkyl group;

each of Y¹ and Y² is independently O, S, or NH;

m is 1, 2, or 3; and

n is 1, 2, 3, or 4.

For example, the compound may have a structure according to Formula (VII),

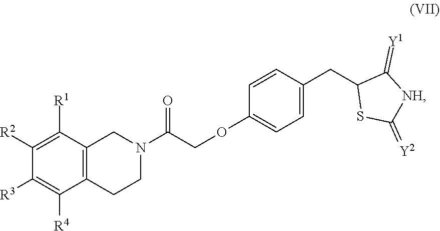

or a pharmaceutically acceptable salt thereof.

In certain embodiments, one or more or each of R¹R², and R⁴ is independently hydrogen or unsubstituted C₁-C₆ alkyl.

In a non-limiting example, R³ is —OH or —OR^{3A}.

Exemplary compounds include

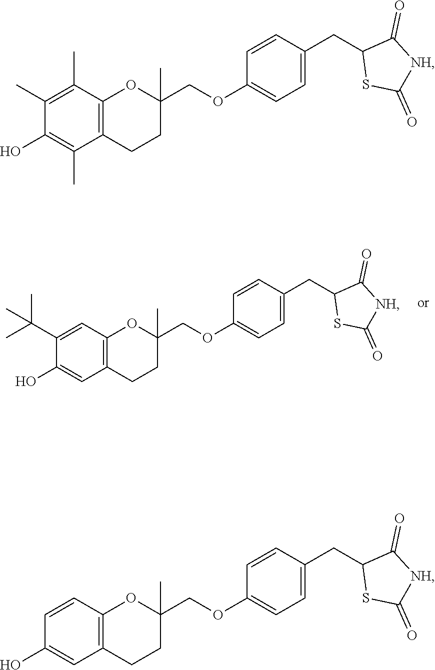 [SMILES: Cc1c(C)c2c(c(C)c1O)CCC(C)(COc1ccc(CC3SC(=O)NC3=O)cc1)O2 ; CC1(COc2ccc(CC3SC(=O)NC3=O)cc2)CCc2cc(O)c(C(C)(C)C)cc2O1 ; "or" ; CC1(COc2ccc(CC3SC(=O)NC3=O)cc2)CCc2cc(O)ccc2O1]

or a pharmaceutically acceptable salt thereof.

Aspects include a method for reducing RPE cell death, reducing the size of GA, inhibiting progression of GA, and/or inhibiting the progression of dry AMD (including, e.g., advanced dry AMD), comprising contacting a RPE cell with a compound, wherein the compound is selected from the group consisting of:

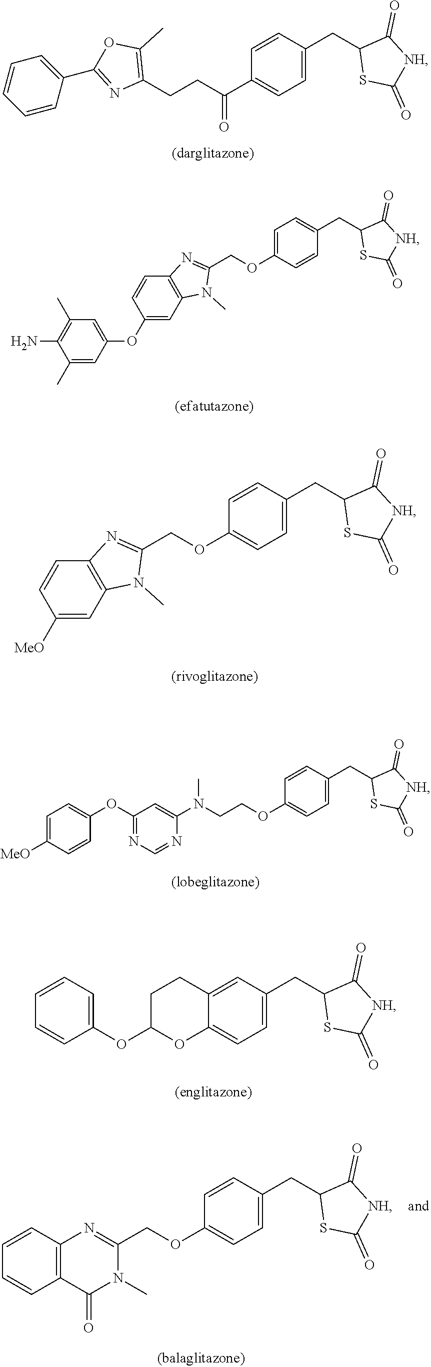

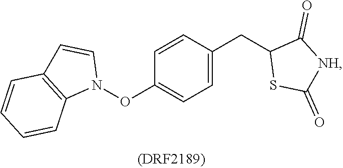 [SMILES: O=C1NC(=O)C(Cc2ccc(On3ccc4ccccc43)cc2)S1]

or a pharmaceutically acceptable salt thereof.

In various embodiments, the compounds described for therapeutic use are purified. Purity is measured by any appropriate standard method, for example, by electrophoresis, column chromatography, thin layer chromatography, liquid chromatography including high-performance liquid chromatography (HPLC) analysis, and mass spectrometry and/or other similar methods. “Purified” also defines a degree of sterility that is safe for administration to a human subject, e.g., lacking infectious or toxic agents. For example, a purified compound is one that is at least 90%, 91%, 92%, 93%, 94%, 95%, 98%, 99%, or 100% (w/w) of the composition by weight.

Small molecule compounds are molecules less than 1000 daltons in molecular mass. In various embodiments, whether an organic compound or peptide, a small molecule compound is between 50-1000 daltons, e.g., less than 750 daltons, 500 daltons, 250 daltons or 100 daltons, in molecular mass. Small molecules include pharmaceutically active organic agents, biological agents, or peptides.

As used herein, “subject”, as used herein, means a mammalian subject (e.g., dog, cat, horse, cow, sheep, goat, monkey, etc.), and particularly human subjects (including both male and female subjects, and including neonatal, infant, juvenile, adolescent, adult and geriatric subjects, and further including various races and ethnicities including, but not limited to, subjects who self-report and/or identify themselves as white (e.g., Caucasian), black (e.g., of African descent), Asian, Native American, and Hispanic).

As used herein, “treatment”, “treat”, and “treating” refer to reversing, alleviating, inhibiting the progress, or delaying the progression of a disorder or disease as described herein. As used herein, “inhibiting” progression in a subject means preventing or reducing the progression in the subject.) In some embodiments, treating a disease or disorder includes ameliorating at least one symptom of the particular disease or disorder, even if the underlying pathophysiology is not affected. In various embodiments, the efficacy of the treatment can be evaluated, e.g., as compared to a standard, e.g., improvement in the value or quality of a parameter (e.g., vision quality, an amount of drusen, and/or the amount or size of GA) as compared to the value or quality of the parameter prior to treatment. As another example, the efficacy of treatment can be evaluated, e.g., as compared to a standard, e.g., slowing progression of the disease as compared to a usual time course for the disease in a cohort that has not been treated or compared to historical data on disease progression. Treating a disease also includes slowing its progress and/or relieving the disease, e.g., causing regression of the disease. In some embodiments, the progressive worsening (e.g., the increasing intensity) of a symptom is slowed, reduced, or halted.

As used herein, “prevention”, “prevent”, and “preventing” describes reducing or eliminating the onset of the symptoms or complications of the disease, condition or disorder. In various embodiments, preventing (or prevention of) a disease includes stopping a disease from occurring in a subject, who may be predisposed to the disease but has not yet been diagnosed as having it. Preventing a disease also includes delaying the onset of the disease. The efficacy of the prevention can be evaluated, e.g., as compared to a standard, e.g., delaying onset of the disease as compared to a usual time of onset for the disease in a cohort that has not been treated or compared to historical data on disease onset.

As used herein, a “symptom” associated with a disorder includes any clinical or laboratory manifestation associated with the disorder, and is not limited to what the subject can feel or observe.

As used herein “an effective amount” refers to an amount that causes relief of symptoms of a disorder or disease as noted through clinical testing and evaluation, subject observation, and/or the like. An “effective amount” can further designate a dose that causes a detectable change in biological or chemical activity. The detectable changes may be detected and/or further quantified by one skilled in the art for the relevant mechanism or process. Moreover, an “effective amount” can designate an amount that maintains a desired physiological state, i.e., reduces or prevents significant decline and/or promotes improvement in the condition of interest. In some embodiments, an “effective amount” can further refer to a therapeutically effective amount.

As used herein, the term “about” in the context of a numerical value or range means ±10% of the numerical value or range recited or claimed, unless the context requires a more limited range.

It is understood that where a parameter range is provided, all integers within that range, and tenths thereof, are also provided by the invention. For example, “0.2-5 mg” is a disclosure of 0.2 mg, 0.3 mg, 0.4 mg, 0.5 mg, 0.6 mg etc. up to and including 5.0 mg.

In the descriptions herein and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristict(s)” of the claimed invention.

The terms “subject,” “patient,” “individual,” and the like as used herein are not intended to be limiting and can be generally interchanged. That is, an individual described as a “patient” does not necessarily have a given disease, but may be merely seeking medical advice.

As used herein, the singular forms “a,” “an,” and “the” include the plural reference unless the context clearly dictates otherwise. Thus, for example, a reference to “a disease,” “a disease state”, or “a nucleic acid” is a reference to one or more such embodiments, and includes equivalents thereof known to those skilled in the art and so forth.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and riot intended to be limiting.

### BRIEF DESCRIPTION OF THE DRAWINGS

FIGS. 1A and 1B are bar graphs showing that oxidized low-density lipoprotein (ox-LDL) induces caspase 1 activation and cell death. In FIG. 1A, ARPE-19 cells were treated with human low-density lipoprotein (LDL) or ox-LDL at 100 μg/ml for 48 hr and a fluorescent inhibitor of caspases (FLICA) probe was used to detect active caspase-1 in cells. In FIG. 1B, ARPE-19 cells were treated for 48 hr with LDL or ox-LDL at 25, 50, or 100 μg/ml. Conditioned media were harvested, and LDH levels were quantified to evaluate cell death. Data=mean SEM, ***P<0.001, one way ANOVA).

FIGS. 2A and 2B are bar graphs showing that the highly selective PPARγ agonist troglitazone significantly suppressed ox-LDL-induced cell death of human primary RPE in a dose-dependent fashion. FIG. 2A shows that PPARγ-selective agonist troglitazone (0.55 μM) significantly suppressed ox-LDL-induced RPE death, whereas fenofibrate (30 μM), a PPARα-selective agonist and bezafibrate (60 μM), a pan-PPARα agonist with selectivity for PPARβ/δ>PPARα>PPARγ treatments did not. FIG. 2B shows that troglitazone applied at doses of 0.65 μM and 0.98 μM significantly decreased cell death in a dose-dependent manner. For both, RPE cells were treated with 500 μg/ml of ox-LDL for 48 hr. Data=mean±SEM, **P<0.01 and ***P<0.001 compared to ox-LDL group (2-tailed unpaired t-test).

FIG. 3 is a bar graph showing PPARγ agonist troglitazone significantly suppressed ox-LDL-induced caspase-1 activation. ARPE-19 cells were serum-starved for 24 hr, treated with ox-LDL at 100 μg/ml with and without troglitazone (1.3 μM) for 48 hr, and a fluorescent inhibitor of caspases (FLICA) probe was used to detect active caspase-1 in cells. Cells were imaged by fluorescent microscope and the numbers of capase-1 positive cells for each treatment group were quantified by a masked investigator. Data=mean±SEM, *P<0.05 and **P<0.01 compared to ox-LDL group (2-tailed unpaired t-test).

FIG. 4 is a bar graph showing PPARγ agonist troglitazone selectively protects RPE from ox-LDL-induced toxicity. Troglitazone significantly suppressed ox-LDL-induced death of primary human RPE cells, but was ineffective in suppressing RPE death induced by chloroquine. Data=mean±SEM, ***P<0.001 compared to ox-LDL group (2-tailed unpaired t-test).

FIG. 5 is a bar graph showing testing different PPARγ agonists for RPE (ARPE-19) protection. PGJ2=15-deoxy-Δ^{12.14}PGJ₂, **P<0.01, ***P<0.001 compared to ox-LDL treatment group.

FIGS. 6A-B are bar graphs showing the effect of different PPARγ agonists on target genes expression in ARPE19 cells (using samples from experiment described in FIG. 5).

FIG. 7 is a bar graph showing testing the effect of different PPAR(α, γ) and RXR antagonists in ox-LDL-induced RPE (ARPE-19) cell death.

### DETAILED DESCRIPTION OF THE INVENTION

GA or late stage dry AMD, is characterized by degeneration and death of the RPE in the macula, resulting in severe, irreversible vision loss. As there is no established treatment for dry AMD and GA, new therapies are needed, and lipid metabolism is a promising area of focus. Although the exact mechanisms underlying the pathogenesis of AMD remain unknown, altered lipid metabolism has been strongly associated with AMD pathogenesis. Lipid-containing drusen are the first sign of AMD, hypercholesterolemia is a risk factor for the disease, and cholesterol-lowering statins may provide some benefit in individuals aged 68 and older, suggesting a role for dysfunctional lipid metabolism in dry AMD.

The RPE plays a key role in lipid homeostasis in the retina. RPE cells express a variety of receptors that are involved in lipid metabolism, including lipid scavenger receptors. Lipids encountered by the RPE originate from photoreceptors and from the choroidal circulation. Apically delivered lipids, including photo-oxidized outer segments of photoreceptor, are normally “cleared” by the RPE to the choroid. The mechanism of lipid deposition in AMD is poorly understood, but the specific location for lipid deposits i.e., underneath the RPE and within Bruch's membrane, suggests that impaired clearance by the RPE is a contributor.

Clinical and experimental observations have shown accumulation of ox-LDL and phospholipids in RPE cells. This accumulation can have important consequences for cellular function, as uptake of oxidized lipids by RPE cells decreases lysosomal protease function. This finding is supported by the observation that RPE cells accumulate ox-LDL in the lysosomes. Given that lysosomal function in RPE cells decreases with age, lysosomal accumulation of oxidized lipids is particularly pronounced in elderly individuals.

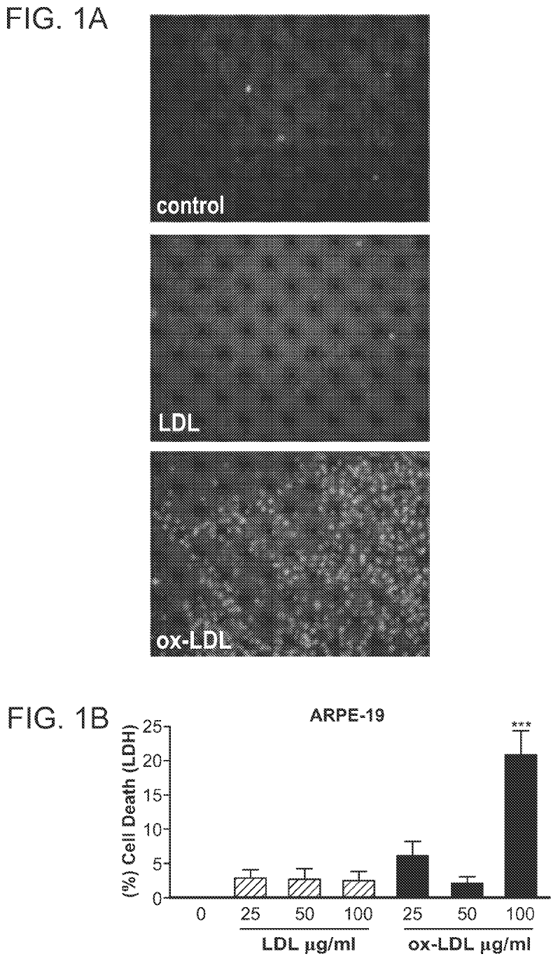

Lysosomal accumulation of ox-LDL has profound impacts on cellular health. In macrophages, accumulation of ox-LDL in the lysosomes leads to crystallization of ox-LDL, which causes lysosomal destabilization, activation of the NLRP3-inflammasome, release of interleukin-1beta (IL-1) and ultimately cell death. Similar effects have been observed in primary human RPE and in ARPE-19 cells. ARPE-19 cells exposed to ox-LDL [but not to native(unoxidized) LDL] released pro-inflammatory cytokines at higher levels, expressed higher levels of NLRP3-inflammasome activation markers and cell death (FIGS. 1A-1B), and similar results were obtained from primary human RPE. Inflammasome activation has also been observed in the RPE cells of human eyes with AMD. The results described herein indicate that oxidized lipids induce inflammosomes and support a role for an ox-LDL-lysosome-inflammasome pathway in the pathogenesis of geographic atrophy and dry AMD. Without wishing to be bound by any scientific theory, the therapeutic methods described herein enhance clearance of oxidized lipids and/or neutralize the toxic effects of internalized oxidized lipids by restoring lysosomal function (as one of the possible mechanisms of action) and thereby suppress the cytotoxic effects of oxidized lipid in dry AMD. The methods suppress oxidized lipid-induced cell death in RPE cells that is associated with the development of dry AMD.

Young and healthy RPE cells effectively process ox-LDL without inducing inflammasome activation. However, RPE cells of adult or elderly individuals are not as efficient to process or clear ox-LDL. Accumulated lipids become oxidized and become deposited on top of RPE, exposing RPE ox-LDL and thereby having clinical consequences such as development of AMD and impaired vision.

One way to limit the cytotoxic impact of ox-LDL is by activating the nuclear receptor peroxisome proliferator-activated receptor gamma (PPARγ). PPARγ increases lipid metabolism and suppresses inflammatory responses. In epithelial cells, PPARγ can accelerate lysosomal maturation and the degradation of oxidized lipids. Activation of PPARγ in an experimental model of ischemic stroke both limits pro-inflammatory interleukin-1 receptor signaling in neuronal cells and facilitates neuroprotection. These observations indicate that activation of PPAR increases clearance of oxidized lipids, reduce cellular inflammation, and reduce cell death in RPE cells, thereby reducing the development of GA and advanced AMD.

PPARγ is expressed in the retina and at high levels in the RPE. The effects of PPARγ agonists including troglitazone on the RPE health upon ox-LDL treatment (i.e. dry AMD) have not been examined prior to the invention. In some embodiments, PPARγ activation by selective agonists increases lysosomal clearance of oxidized lipids. The data described herein indicate that PPARγ agonist troglitazone reduces inflammasome activation, proinflammatory cytokine release and cell death in RPE. Thus, PPAR agonists are useful for treatment for late stage dry AMD and GA.

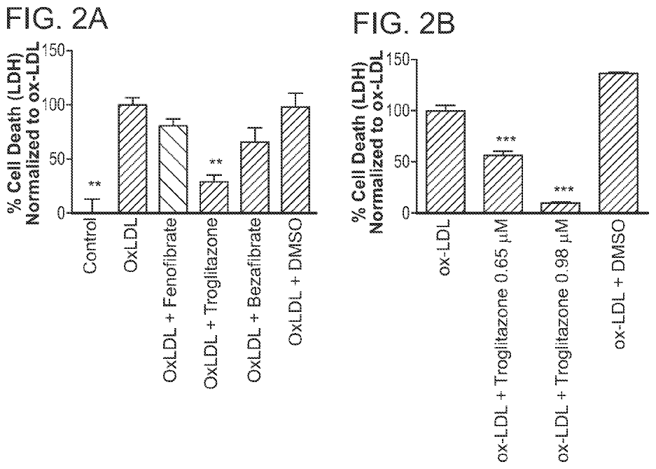

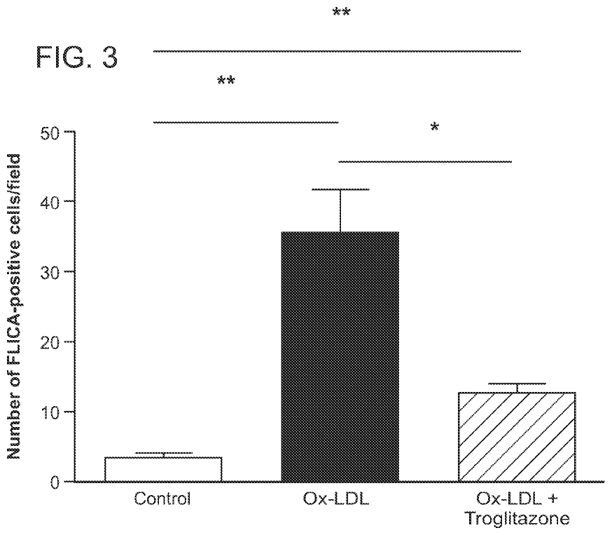

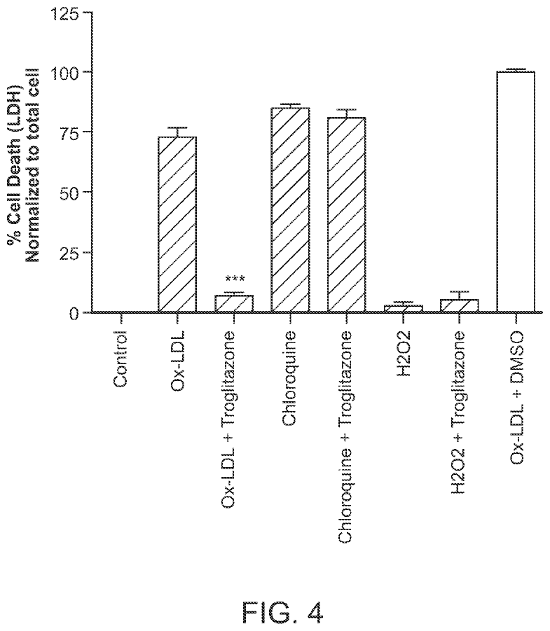

The data show that PPARγ agonist troglitazone, but not agonists for PPARα or PPARβ/δ, significantly reduced ox-LDL-induced cell death in a dose-dependent manner in both human primary RPE and APRE-19 cells (FIGS. 2A-2B). Furthermore, treatment of ARPE-19 cells with PPARγ agonist troglitazone significantly suppressed ox-LDL-induced caspase 1 activation, indicating that PPARγ activation suppresses ox-LDL-induced activation of the inflammasome and therefore cell death of RPE (FIG. 3). Activation of PPARγ in RPE cells by troglitazone inhibited ox-LDL induced cell death, but not cell death induced by other toxic insults such as chloroquine indicating that the PPARγ protective pathway in RPE is specific to oxidized lipids-induced cellular degeneration (FIG. 4). These data indicate that PPARγ selective agonists such as troglitazone and analogues thereof are useful to mitigate the retinal and RPE damage associated with dry AMD. Such compounds are used to treat dry AMD by ocular delivery in the form of intraocular injection, periorbital injection or topical application, to limit high levels of systemic exposure, which is associated with acute hepatic and cardiac negative side effects in patients.

Thus, the agonist or agonists used in the methods are characterized by K_{d} that is at least about 0.0, 0.00001, 0.0001, 0.001, 0.01, 0.01, 0.1, 0.5, 1, 5, 10, 50, 100, 200, 300, 400, 500, 750, or 1000 μM higher for PPAR-α pr β/δ than for PARRγ in distilled water, cell culture media, or phosphate buffered saline. By the K_{d} of 0.00 higher for PPAR-α or β/δ than for PPARγ means that the agonist can bind to PPAR-α or β/δ forms equally well compared to the PPARγ form. In another example, the EC₅₀ of the agonist is at least about 0.0, 0.00001, 0.0001, 0.001, 0.01, 0.1, 0.5, 1, 5, 10, 50, 100, 200, 300, 400, 500, 750, or 1000 μM higher for PPAR-α or β/δ than for PPARγ based on induction of PPARγ-targeted genes expression in cells or similar functional assays.

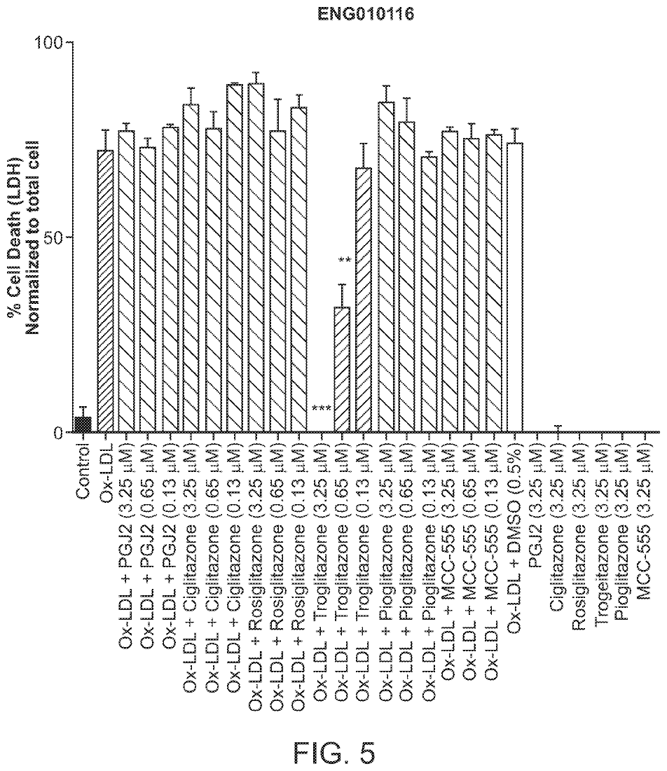

FIG. 5 shows the effect of testing different PPARγ agonists for RPE (ARPE-19) protection. Only troglitazone (at 0.65 and 3.25 μM) significantly protected RPE cell from ox-LDL-induced cell death, and none of the other PPARγ agonists demonstrated detectable protection of RPE from ox-LDL-induced cell death. The compound 15-deoxy-Δ^{12,14}-Prostaglandin J₂(PGJ2), considered a natural ligand, EC₅₀: 2-7 μM. The following agonists, in addition to PGJ2, were tested:
Ciglitazone, TZD class agonist, EC₅₀: 3 μM.
Rosiglitazone, TZD class agonist, EC₅₀: 30-100 nM.
Troglitazone, TZD class agonist, EC₅₀: 0.55-0.78 μM.
Pioglitazone, TZD class agonist, EC₅₀: 500-600 nM.
MCC-555, TZD class agonist, structure homolog of rosiglitazone, EC50 lower than that of rosiglitazone.

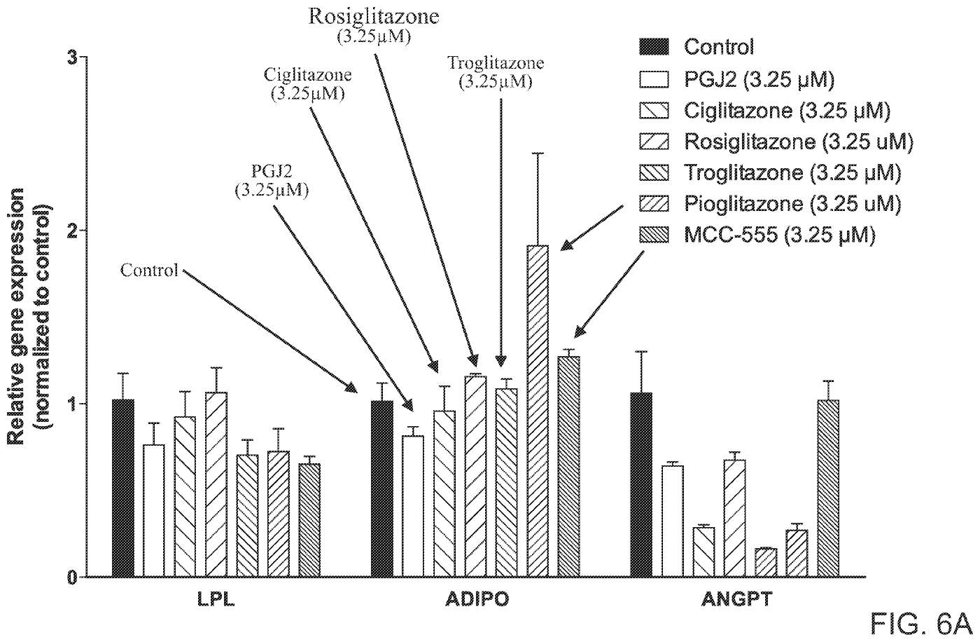

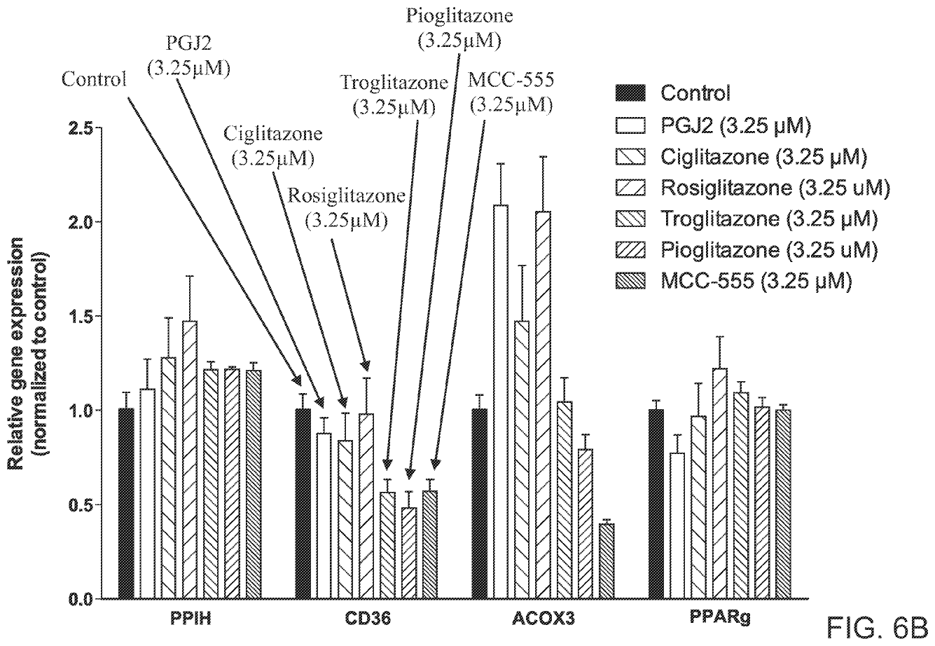

FIGS. 6A-B show the effect of different PPARγ agonists on target genes expression in ARPE19 cells (using samples from experiment shown in FIG. 5 above). FIGS. 6A-6B show data from a 48-hour treatment. Focusing on troglitazone (blue) vs. rosiglitazone (red) at 48 hours, the expression levels of two PPARγ target genes are differentially affected by these two different agonists: ANGPT (angiopoietin-like-4), ACOX3 (peroxisomal acyl-coenzyme A oxidase 3), suggesting differential function of troglitazone and rosiglitazone in modulating PPARγ activity in RPE cells.

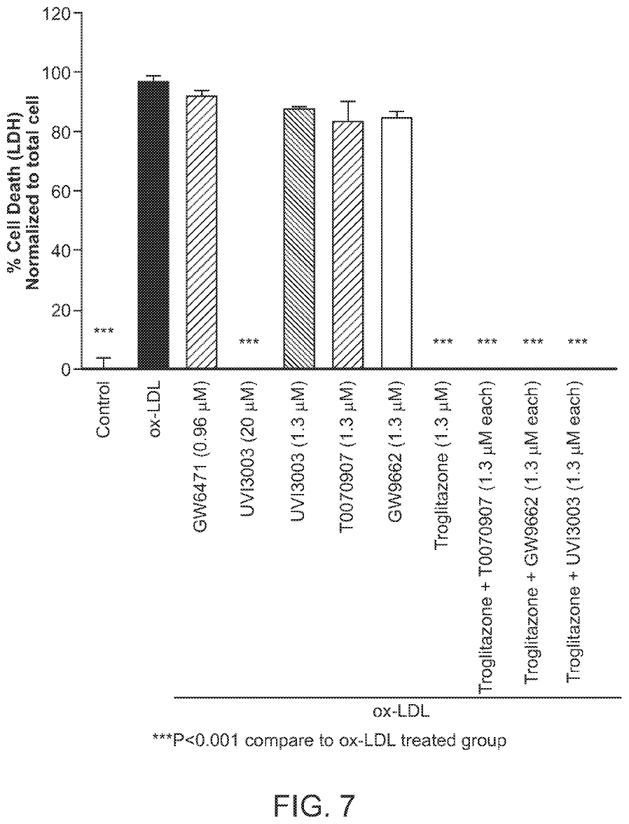

FIG. 7 shows testing the effect of different PPAR(α, γ) and RXR antagonists in ox-LDL-induced RPE (ARPE-19) cell death. PPARα antagonist GW6471 had no detectable effect in RPE protection, and RXR antagonist UV13003 at high dose (20 μM) also significantly protected RPE from ox-LDL-induced death (both at about 100%). PPARγ antagonists T0070907 and GW9662 did not have any detectable protection effect for RPE, and they also did not affect the protection effect by PPARγ agonist troglitazone on RPE, troglitazone (1.3 μM) alone or in combination with other PPARγ antagonists (T0070907 and GW9662) significantly protected the RPE against ox-LDL-induced cell death. The following antagonists were tested.
GW6471, PPARα antagonist, IC50: 0.24 μM.
UVI 3003, RXR antagonist, IC50<1 μM
T0070907, PPARγ antagonist, IC50<1 μM.
GW9662, PPARγ antagonist, IC50<0.1 μM.

The structures of these antagonists are as follows:

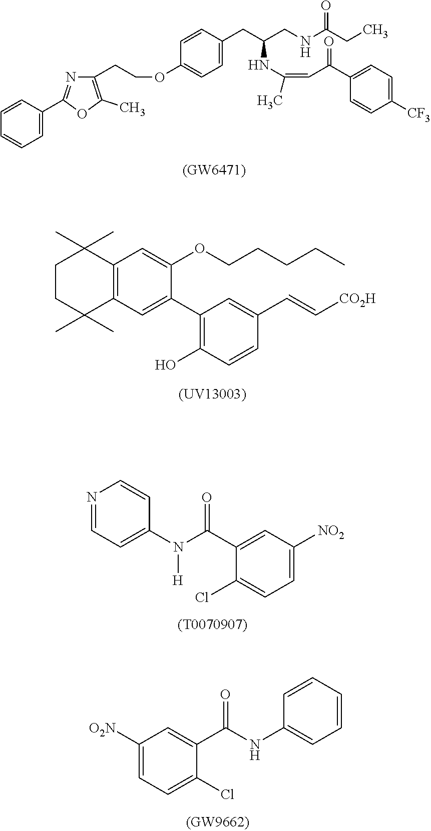 [SMILES: CCC(=O)NC[C@H](Cc1ccc(OCCc2nc(-c3ccccc3)oc2C)cc1)N/C(C)=C\C(=O)c1ccc(C(F)(F)F)cc1 ; CCCCCOc1cc2c(cc1-c1cc(/C=C/C(=O)O)ccc1O)C(C)(C)CCC2(C)C ; O=C(Nc1ccncc1)c1cc([N+](=O)[O-])ccc1Cl ; O=C(Nc1ccccc1)c1cc([N+](=O)[O-])ccc1Cl]

### PPARγ Selective Agonists: Troglitazone and Analogues Thereof

Thiazolidinediones or TZDs act by activating PPARs, a group of nuclear receptors, with greatest specificity for PPARγ (gamma). The endogenous ligands for these receptors are free fatty acids (FFAs) and eicosanoids. When activated, the receptor binds to DNA in complex with the RXR, another nuclear receptor, increasing transcription of a number of specific genes and decreasing transcription of others.

Compounds suitable for use in the methods described herein include those described in U.S. Pat. No. 5,602,133 issued Feb. 11, 1997, which is incorporated herein by reference in its entirety. In embodiments, a compound has a structure according to the following formula,

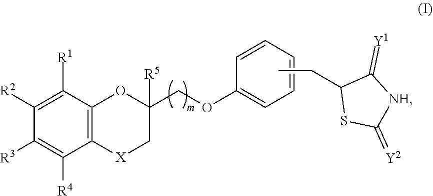

or a pharmaceutically acceptable salt thereof, wherein

each of R¹ and R² is independently hydrogen, C₁-C₆ alkyl, or a C₁-C₆ alkoxy group, or R¹ and R² combine to form a —O(CH₂)ₙO— group;

R³ is hydrogen, —R^{3A}, —OH, OR^{3A}, —OC(═O )R^{3A}, or —OC(═O )OR^{3A};

R^{3A} is C₁-C₆ alkyl, C₆-C₁₀ aryl, 5- to 6-membered heteroaryl, C₃-C₈ cycloalkyl, or a three- to eight-membered heterocycloalkyl;

R⁴ is a hydrogen atom or a C₁-C₆ alkyl group;

R⁵ is a hydrogen atom or a C₁-C₆ alkyl group;

X is —CH₂—, —C(═O)—, or —CHOR^{X}—;

R^{X} is C₁-C₆ alkyl, C₆-C₁₀ aryl, 5- to 6-membered heteroaryl, C₃-C₈ cycloalkyl, or a three- to eight-membered heterocycloalkyl;

each of Y¹ and Y² is independently O, S, or NH;

m is 1, 2, or 3; and

n is 1, 2, 3, or 4.

In embodiments, X is —CH₂—. In embodiments, Y¹ and Y² are each O. In embodiments, R¹, R², and R⁴ are each independently hydrogen or unsubstituted C₁-C₆ alkyl. In embodiments, R³ is hydrogen, —OH, or —OR^{3A}. In embodiments, R⁵ is hydrogen or unsubstituted C₁-C₆ alkyl, In embodiments, m is 1.

In embodiments, the compound has a structure according to Formula (II),

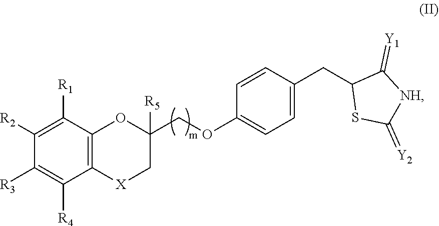

or a pharmaceutically acceptable salt thereof, wherein each of R¹-R⁵₅ X, m, Y¹, and Y² are as defined for Formula (I).

In embodiments, X is —CH₂—. In embodiments, Y ¹ and Y² are each O. In embodiments, R¹, R², and R⁴ are each independently hydrogen or unsubstituted C₁-C₆ alkyl. In embodiments, R³ is hydrogen, —OH, or —OR^{3A}. In embodiments, R⁵ is hydrogen or unsubstituted C₁-C₆ alkyl. In embodiments, m is 1.

In embodiments, the compound has a structure according to Formula (III).

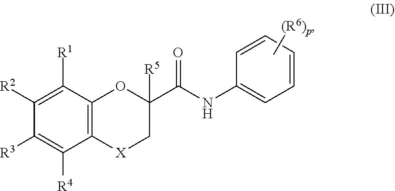

or a pharmaceutically acceptable salt thereof, wherein:

each of R¹ and R² is independently hydrogen, C₁-C₆ alkyl, or a C₁-C₆ alkoxy group, or R¹ and R² combine to form a —O(CH₂)ₙO— group;

R³ is hydrogen, —R^{3A}, —OR^{3A}, —OC(═O)R^{3A}, or —OC(═O)OR^{3A};

R^{3A} is C₁-C₆ alkyl, C₆-C₁₀ aryl, 5- to 6-membered heteroaryl, C₃-C₈ cycloalkyl, or a three- to eight-membered heterocycloalkyl;

R⁴ is a hydrogen atom or a C₁-C₆ alkyl group;

R⁵ is a hydrogen atom or a C₁-C₆ alkyl group;

R⁶, when present, is independently selected from halogen, —CN, —NO₂, C₁-C₆ alkyl, C₆-C₁₀ aryl, 5- to 6-membered heteroaryl, C₃-C₈ cycloalkyl, and a three- to eight-membered heterocycloalkyl;

X is —CH₂—, or —C(═O)—, —CHOR^{X}—;

each R^{X} is independently C₁-C₆ alkyl, C₆-C₁₀ aryl, 5- to 6-membered heteroaryl, C₃-C₈ cycloalkyl, or a three- to eight-membered heterocycloalkyl;

each of Y¹ and Y² is independently O, S, or NH;

n is 1, 2, 3, or 4; and

p is 0, 1, 2, 3, 4, or 5.

In embodiments, X is —CH₂—. In embodiments, R¹, R², and R⁴ are each independently hydrogen or unsubstituted C₁-C₆ alkyl. In embodiments, R³ is hydrogen, —OH, or —OR^{3A}. In embodiments, R⁵ is hydrogen or unsubstituted C₁-C₆ alkyl. In embodiments, p is 1 or 2. In embodiments, one R⁶, when present is C₁-C₆ alkyl substituted with C₆-C₁₀ aryl, 5- to 6-membered heteroaryl, C₃-C₈ cycloalkyl, or a three- to eight-membered heterocycloalkyl.

In embodiments, the compound has a structure according to Formula (IV),

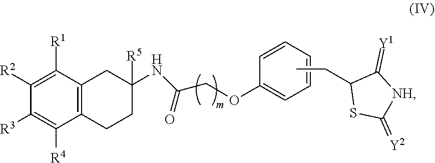

or a pharmaceutically acceptable salt thereof, wherein:

each of R¹ and R² is independently hydrogen, C₁-C₆ alkyl, or a C₁-C₆ alkoxy group, or R¹ and R² combine to form a —O(CH₂)ₙO— group;

R³ is hydrogen, —R^{3A}, —OH, —OR^{3A}, —OC(═O)R^{3A}, or —OC(═O)OR^{3A};

R^{3A} is C₁-C₆ alkyl, C₆-C₁₀ aryl, 5- to 6-membered heteroaryl, C₃-C₈ cycloalkyl, or a three- to eight-membered heterocycloalkyl;

R⁴ is a hydrogen atom or a C₁-C₆ alkyl group;

R⁵ is a hydrogen atom or a C₁-C₆ alkyl group;

each of Y¹ and Y² is independently O, S, or NH;

m is 1, 2, or 3; and

n is 1, 2, 3, or 4.

In embodiments Y¹ and Y² are each O. In embodiments, R¹, R², and R⁴ are each independently hydrogen or unsubstituted C₁-C₆ alkyl. In embodiments, R³ is hydrogen, —OH, or —OR^{3A}. In embodiments, R⁵ is hydrogen or unsubstituted C₁-C₆ alkyl. In embodiments, m is 1.

In embodiments, the compound has a structure according to Formula (V),

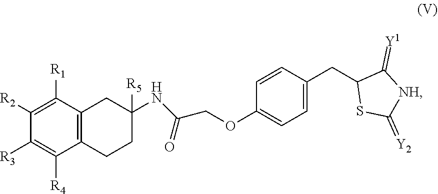

or a pharmaceutically acceptable salt thereof, wherein each of R¹-R⁵, Y¹, and Y² are as defined for Formula (IV).

In embodiments, Y¹ and Y² are each O. In embodiments, R¹, R², and R⁴ are each independently hydrogen or unsubstituted C₁-C₆alkyl. In embodiments, R³ is hydrogen, —OH, or —OR^{3A}. In embodiments, R⁵ is hydrogen or unsubstituted C₁-C₆ alkyl.

In embodiments, the compound has a structure according to Formula (VI),

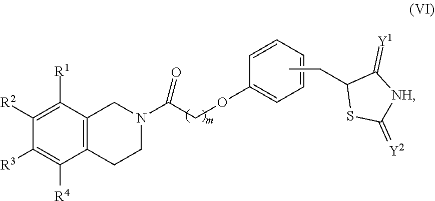

or a pharmaceutically acceptable salt thereof, wherein

each of R¹ and R² is independently hydrogen, C₁-C₆ alkyl, or a C₁-C₆ alkoxy group, or R¹ and R² combine to form a —O(CH₂)ₙO— group;

R³ is hydrogen, —R^{3A}, —OH, —OR^{3A}, —OC(═O)R^{3A}, or —OC(═O)OR^{3A},

R^{3A} is C₁-C₆ alkyl, C₆-C₁₀ aryl, 5- to 6-membered heteroaryl, C₃-C₈ cycloalkyl, or a three- to eight-membered heterocycloalkyl,

R⁴ is a hydrogen atom or a C₁-C₆ alkyl group;

each of Y¹ and Y² is independently O, S, or NH;

m is 1, 2, or 3; and

n is 1, 2, 3, or 4.

In embodiments, the compound comprises a thiazolidinedione (TZD) domain or a derivative thereof that can activate PPARs, for example, by having substantial specificity for PPARγ (gamma). In embodiments, the thiazolidinedione (TZD) domain or its derivative may be represented by the structure of

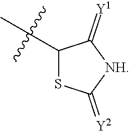

In embodiments, Y¹ and Y² may be same or different and independently O, S, or NH. In embodiments Y¹ and Y¹ are each O, and the thiazolidinedione moiety may have the structure of

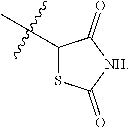 [SMILES: *C1SC(=O)NC1=O]

In embodiments, R¹, R², and R⁴ are each independently hydrogen or unsubstituted C₁-C₆ alkyl. In embodiments, R³ is hydrogen, —OH, or —OR^{3A}. In embodiments, m is 1.

In embodiments, the compound has a structure according to Formula (VII),

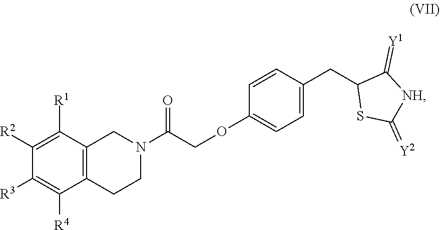

or a pharmaceutically acceptable salt thereof, wherein R¹-R⁴, Y¹, and Y² are as defined herein for Formula (VI).

In embodiments, Y¹ and Y² are each O. In embodiments, R¹, R², and R⁴ are each independently hydrogen or unsubstituted C₁-C₆ alkyl. In embodiments, R³ is hydrogen, —OH, or —OR^{3A}.

In embodiments, the compound is

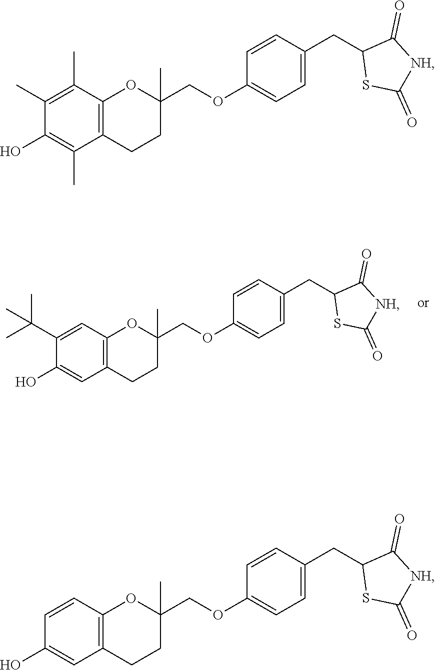 [SMILES: Cc1c(C)c2c(c(C)c1O)CCC(C)(COc1ccc(CC3SC(=O)NC3=O)cc1)O2 ; CC1(COc2ccc(CC3SC(=O)NC3=O)cc2)CCc2cc(O)c(C(C)(C)C)cc2O1 ; "or" ; CC1(COc2ccc(CC3SC(=O)NC3=O)cc2)CCc2cc(O)ccc2O1]

or a pharmaceutically acceptable salt thereof. In embodiments, the compound is

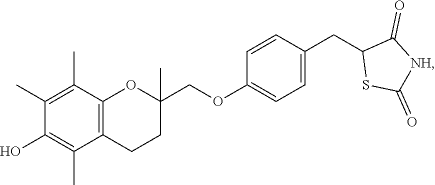 [SMILES: Cc1c(C)c2c(c(C)c1O)CCC(C)(COc1ccc(CC3SC(=O)NC3=O)cc1)O2]

or a pharmaceutically acceptable salt thereof.

In embodiments, the compound is selected from the group consisting of:

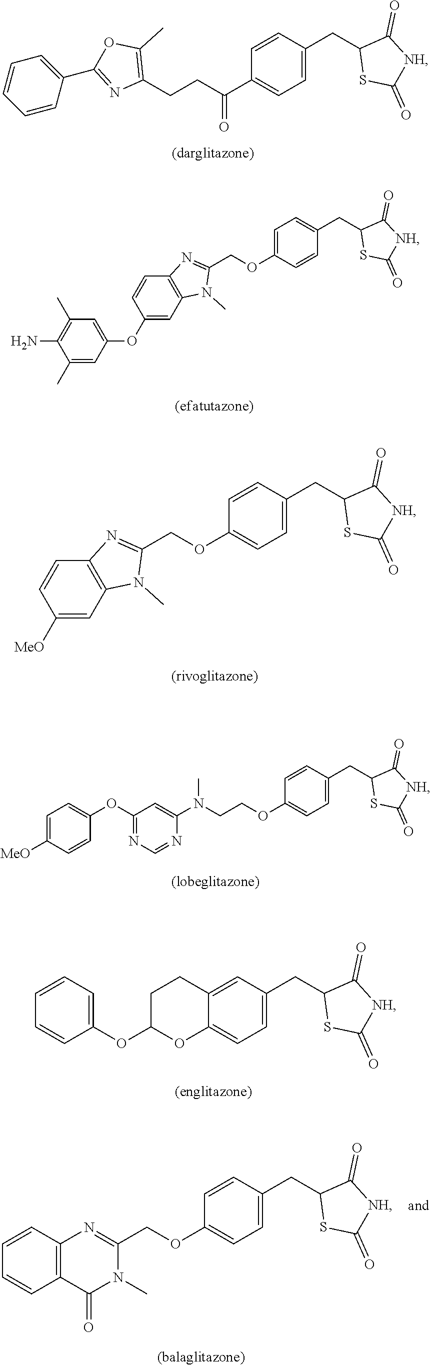

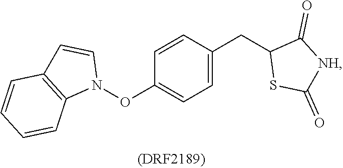 [SMILES: O=C1NC(=O)C(Cc2ccc(On3ccc4ccccc43)cc2)S1]

or a pharmaceutically acceptable salt thereof.

Compounds for use in the methods described herein can be prepared according to methods known in the art. See, e.g., U.S. Pat. No. 5,602,133, and Sugawara et al., Hypertension Research, 24(3), 229-233, 2001, each of which is herein incorporated by reference in its entirety.

The term “alkyl,” by itself or as part of another substituent,ns, unless otherwise stated, a straight (i.e., unbranched) or branched carbon chain (or carbon), or combination thereof, which may be fully saturated, mono- or polyunsaturated and can include mono-, di- and multivalent radicals, having the number of carbon atoms designated (i.e., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. Art unsaturated alkyl group is one having one or more double bonds or triple bonds. Examples of unsaturated alkyl groups include, but are not limited to, vinyl, 2-propenyl, crotyl, 2-isopentenyl., 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobermofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl , 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5 isoctuinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Chemical groups described herein may be unsubstituted or substituted with one or more substituent groups (e.g., 1, 2, 3, 4, or 5 substituent groups). A “substituent group,” as used herein, means a group selected from the following moieties:
(A) oxo, halogen, —CF₃, —CN, —OH, —NH₂, —COOH —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, heteroaryl, substituted with at least one substituent selected from:
   (i) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, heteroaryl, substituted with at least one substituent selected from:
      (a) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl heteroaryl, substituted with at least one substituent selected from: oxo, halogen, —CF₃—CN, —OH, —NH₂, —COON, —CONH₂, —NO₂, —SH, —SO₃H, —SO₄H, —SO₂NH₂, —NHNH₂, —ONH₂, NHC═(O)NHNH₂, —NHC═(O) NH₂, —NHSO₂H, —NHC═(O)H, —NHC(O)—OH, —NHOH, —OCF₃, —OCHF₂, unsubstituted unsubstituted heteroalkyl, unsubstituted cycloalkyl, unsubstituted heterocycloalkyl, unsubstituted aryl, unsubstituted heteroaryl.

As used herein, a “pharmaceutically acceptable salt” is meant to include a salt of the indicated compound(s) that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids such as hydrochloric, hydrobromic, nitric, carbonic, monohydrogencarbonic, phosphoric, monohydrogenphosphoric., dihydrogenphosphoric, sulfuric, monohydrogensulfinic, hydriodic, or phosphorous acids and the like, as well as the salts derived from relatively nontoxic organic acids like acetic, propionic, isobutyric, maleic., malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

Thus, in various embodiments, the compounds of the present invention may exist as salts, such as with pharmaceutically acceptable acids. The present invention includes such salts. Non-limiting examples of such salts include hydrochlorides, hydrobromides, phosphates, sulfates, methanesulfonates, nitrates, maleates, acetates, citrates, fumarates, proprioniates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

In some embodiments, the neutral forms of the compounds are regenerated by contacting the salt with a base or acid and isolating the parent compound in the conventional manner. The parent form of the compound may differ from the various salt forms in certain physical properties, such as solubility in polar solvents.

“Pharmaceutically acceptable excipient” or “pharmaceutically acceptable carrier” refers to a substance that aids the administration of an active agent to a subject and can be included in a composition of the present invention without causing a significant adverse toxicological effect on the subject. In some embodiments, an excipient aids the absorption of an active agent by a subject. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, and quaternary ammonium (methyl iodide, ethyl iodide, and the like) salts.

### Exemplary Pharmaceutical Formulations

Dosages, formulations, dosage volumes, regimens, and methods for agonizing PPARγ can vary. Thus, minimum and maximum effective dosages vary depending on the method of administration.

In various embodiments, a composition comprising a PPARγ agonist may be administered only once or multiple times. For example, a PPARγ agonist may be administered using a method disclosed herein at least about once, twice, three times, four times, five times, six times, or seven times per day week, month, or year. In some embodiments, a composition comprising a PPARγ agonist is administered once per month. In certain embodiments, the composition is administered once per month via intravitreal injection. In various embodiments, such as embodiments involving eye drops, a composition is self-administered.

In some embodiments, a formulation is in the form of a solid, a paste, an ointment, a gel, a liquid, an aerosol, a mist, a polymer, a contact lens, a film, an emulsion, or a suspension. In certain embodiments, the formulations are administered topically, the composition is delivered and directly contacts the eye. In various embodiments, the composition is present at a concentration of 0.01-50% (weight/volume). For example, the inhibitory composition may be present at concentrations of 1% (weight/volume), 10% (weight/volume), 20% (weight/volume), 25% (weight/volume), 30% (weight/volume), 40% (weight/volume), 50% (weight/volume), or any percentage point in between. In some embodiments, the method does not involve systemic administration or planned substantial dissemination of the composition to non-ocular tissue.

Optionally, the composition further contains a pharmaceutically-acceptable carrier. Exemplary pharmaceutical carriers include, but are not limited to, compounds selected from the group consisting of a physiological acceptable salt, poloxamer analogs with carbopol, carbopol/hydroxypropyl methyl cellulose (HPMC), carbopol-methyl cellulose, a mucolytic agent, carboxymethylcellulose (CMC), hyaluronic acid, cyclodextrin, and petroleum. In one embodiment, the mucolytic agent is N-acetyl cysteine.

In some embodiments, a PPARγ agonist (e.g., a pharmaceutical composition comprising a PPARγ agonist) may be administered locally, e.g., as a topical eye drop, peri-ocular injection (e.g., sub-tenon), intraocular injection, intravitreal injection, retrobulbar injection, intraretinal injection, subconjunctival injection, or using iontophoresis, or peri-ocular devices which can actively or passively deliver drug.

In certain embodiments, pharmaceutical formulations adapted for topical administration may be formulated as aqueous solutions, ointments, creams, suspensions, lotions, powders, solutions, pastes, gels, sprays, aerosols, liposomes, microcapsules, microspheres, or oils.

In various embodiments, pharmaceutical formulations adapted for topical administrations to the eye include eye drops wherein a PPARγ agonist is dissolved or suspended in a suitable carrier, especially an aqueous solvent. In some embodiments, a composition to be administered to the eye has an ophthalmically compatible pH and osmolality. The term “ophthalmically acceptable vehicle” means a pharmaceutical composition having physical properties (e.g., pH and/or osmolality) that are physiologically compatible with ophthalmic tissues.

In certain embodiments, an ophthalmic composition of the present invention is formulated as sterile aqueous solutions having an osmolality of from about 200 to about 400 milliosmoles/kilogram water (“mOsm/kg”) and a physiologically compatible pH. The osmolality of the solutions may be adjusted by means of conventional agents, such as inorganic salts (e.g., NaCl), organic salts (e.g., sodium citrate), polyhydric alcohols (e.g., propylene glycol or sorbitol) or combinations thereof.

In various embodiments, the ophthalmic formulations may be in the form of liquid, solid or semisolid dosage form. In some embodiments, the ophthalmic formulations of may comprise, depending on the final dosage form, suitable ophthalmically acceptable excipients. In certain embodiments, the ophthalmic formulations are formulated to maintain a physiologically tolerable pH range. In various embodiments, the pH range of the ophthalmic formulation is in the range of from about 5 to about 9. In some embodiments, pH range of the ophthalmic formulation is in the range of from about 6 to about 8, or is about 6.5, about 7, or about 7.5.

In certain embodiments, the composition is in the form of an aqueous solution, such as one that can be presented in the form of eye drops. By means of a suitable dispenser, a desired dosage of the active agent can be metered by administration of a known number of drops into the eye, such as by one, two, three, four, or five drops.

In various embodiments, one or more ophthalmically acceptable pH adjusting agents and/or buffering agents is included in a composition, including acids such as acetic, boric, citric, lactic, phosphoric, and hydrochloric acids; bases such as sodium hydroxide, sodium phosphate, sodium borate, sodium citrate, sodium acetate, and sodium lactate; and buffers such as citrate/dextrose, sodium bicarbonate, and ammonium chloride. Such acids, bases, and buffers can be included in an amount required to maintain pH of the composition in an ophthalmically acceptable range. In some embodiments, one or more ophthalmically acceptable salts is included in the composition in an amount sufficient to bring osmolality of the composition into an ophthalmically acceptable range. Such salts include those having sodium, potassium, or ammonium cations and chloride, citrate, ascorbate, borate, phosphate, bicarbonate, sulfate, thiosulfate, or bisulfite anions.

Pharmaceutical compositions for ocular delivery also include in situ gellable aqueous compositions. Such compositions may comprise a gelling agent in a concentration effective to promote gelling upon contact with the eye or with lacrimal fluid. Suitable gelling agents include but are not limited to thermosetting polymers. The term “in situ gellable” as used herein includes not only liquids of low viscosity that form gels upon contact with the eye or with lacrimal fluid, but also includes more viscous liquids such as semi-fluid and thixotropic gels that exhibit substantially increased viscosity or gel stiffness upon administration to the eye. See, for example, Ludwig, Adv. Drug Deliv. Rev. 3; 57:1595-639 (2005), the entire content of which is incorporated herein by reference.

### Drug Delivery by Contact Lens

Provided herein is a contact lens comprising a PPARγ agonist. For example, a PPARγ agonist may be incorporated into or coated onto the lens. In some embodiments, a PPARγ agonist is chemically bound or physically entrapped by the contact lens polymer. Alternatively, a color additive is chemically bound or physically entrapped by the polymer composition that is released at the same rate as a PPARγ agonist, such that changes in the intensity of the color additive indicate changes in the amount or dose of PPARγ agonist remaining bound or entrapped within the polymer. Alternatively, or in addition, an ultraviolet (UV) absorber is chemically bound or physically entrapped within the contact lens polymer. In certain embodiments, the contact lens is either hydrophobic and/or hydrophilic.

Exemplary materials used to fabricate a hydrophobic lens to deliver a composition disclosed herein include, but are not limited to, amefocon A, amsilfocon A, aquilafocon A, arfocon A, cabufocon A, cabufocon B, carbosilfocon A, crilfocon A, crilfocon B, dimefocon A, enflufocon A, enflofocon B, erifocon A, flurofocon A, flusilfocon A, flusilfocon B, flusilfocon C, fusilfocon D, flusilfocon E, hexafocon A, hofocon A, hybufocon A, itabisfluorofocon A, itafluorofocon A, itafocon A, itafocon B, kolfocon A, kolfocon B, kolfocon C, kolfocon D, lotifocon A, lotifocon B, lotifocon C, melafocon A, migafocon A, nefocon A, nefocon B, nefocon C, onsifocon A, oprifocon A, oxyfluflocon A, paflufocon B, paflufocon C, paflufocon D, paflufocon E, paflufocon F, pasifocon A, pasifocon B, pasifocon C, pasifocon D, pasifocon F, pemufocon A, porofocon A, porofocon B, roflufocon A, roflufocon B, roflufocon C, roflufocon D, roflufocon E, rosilfocon A, satafocon A, siflufocon A, silafocon A, sterafocon A, sulfocon A, sulfocon B, telafocon A, tisilfocon A, tolofocon A, trifocon A, unifocon A, vinafocon A, and wilofocon A.

Exemplary materials used to fabricate a hydrophilic lens with means to deliver a composition disclosed herein include, but are not limited to, abafilcon A, acofilcon A, acofilcon B, acquafilcon A, alofilcon A, alphafilcon A, amfilcon A, astifilcon A, atlafilcon A, balafilcon A, bisfilcon A, bufilcon A, comfilcon A, crofilcon A, cyclofilcon A, darfilcon A, deltafilcon A, deltafilcon B, dimefilcon A, droxfilcon A, elastofilcon A, epsilfilcon A, esterifilcon A, etafilcon A, focofilcon A, galyfilcon A, genfilcon A, govafilcon A, hefilcon A, hefilcon B, hefilcon C, hilafilcon A, hilafilcon B, hioxifilcon A, hioxifilcon B, hioxifilcon C, hydrofilcon A, lenefilcon A, licryfilcon A, licryfilcon B, lidolficon A, lidofilcon B, lotrafilcon A, lotrafilcon B, mafilcon A, mesafilcon A, methafilcon B, mipafilcon A, nelfilcon A, netrafilcon A, ocufilcon A, ocufilcon B, C, ocufilcon D, ocufilcon E, ofilcon A, omafilcon A, oxyfilcon A, pentafilcon A, perfilcon A, pevafilcon A, phemfilcon A, polymacon, senofilcon A, silafilcon A, siloxyfilcon A, surfilcon A, tefilcon A, tetrafilcon A, trilfilcon A, vifilcon A, vifilcon B, and xylofilcon A.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A method of reducing retinal pigment epithelium (RPE) cell death, reducing the size of geographic atrophy (GA), inhibiting progression of GA, or inhibiting the progression of dry age-related macular degeneration (AMD) in a subject, comprising contacting RPE cells with a peroxisome proliferator-activated receptors-gamma (PPARγ) selective agonist or a retinoid X receptor (RXR) antagonist.

2. The method of claim 1, wherein said agonist comprises troglitazone having a structure of 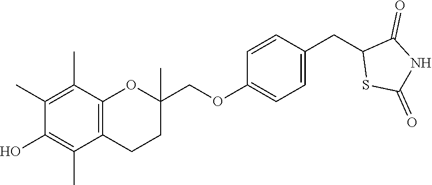 [SMILES: Cc1c(C)c2c(c(C)c1O)CCC(C)(COc1ccc(CC3SC(=O)NC3=O)cc1)O2]
or an analog, or pharmaceutically acceptable salt thereof.

3. The method of claim 1, wherein said agonist comprises a compound having a structure according to Formula (I), 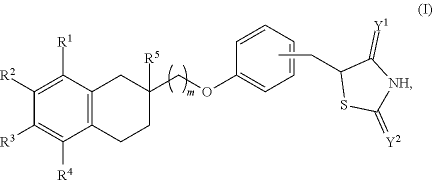
or
Formula (II), 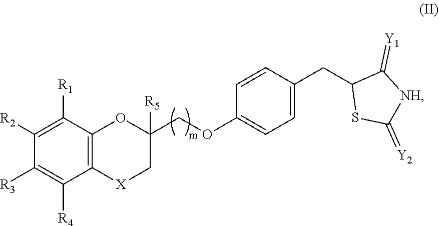
or a pharmaceutically acceptable salt thereof,
wherein:
each of R¹ and R² is independently hydrogen, C₁-C₆ alkyl, or a C₁-C₆ alkoxy group, or R¹ and R² are joined to form a —O(CH₂)ₙO— group;
R³ is hydrogen, —R^{3A}, —OH, —OR^{3A}, —OC(═O)R^{3A}, or —OC(═O)OR^{3A},
R^{3A} is C₁-C₆ alkyl, C₆-C₁₀ aryl, 5- to 6-membered heteroaryl, C₃-C₈ cycloalkyl, or a three- to eight-membered heterocycloalkyl;
R⁴ is a hydrogen atom or a C₁-C₆ alkyl group;
R⁵ is a hydrogen atom or a C₁-C₆ alkyl group;
X is —CH₂—, —C(═O)—, or —CHOR^{X}—;
R^{X} is C₁-C₆ alkyl, C₆-C₁₀ aryl, 5- to 6-membered heteroaryl, C₃-C₈ cycloalkyl, or a three- to eight-membered heterocycloalkyl;
each of Y¹ and Y² is independently O, S, or NH;
m is 1, 2, or 3; and
n is 1, 2, 3, or 4.

4. (canceled)

5. The method of claim 3, wherein X is —CH₂—, R⁵ is hydrogen or unsubstituted C₁-C₆ alkyl, each of R¹, R², and R⁴ is independently hydrogen or unsubstituted C₁-C₆ alkyl, or R³ is —OH or —OR^{3A}.

6-8. (canceled)

9. The method of claim 3, wherein said compound comprises: 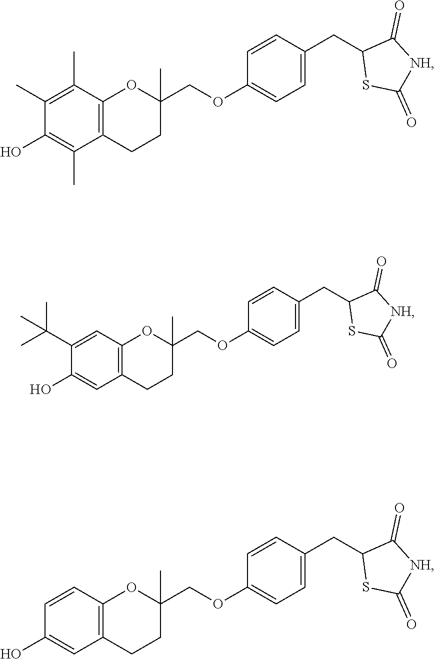 [SMILES: Cc1c(C)c2c(c(C)c1O)CCC(C)(COc1ccc(CC3SC(=O)NC3=O)cc1)O2 ; CC1(COc2ccc(CC3SC(=O)NC3=O)cc2)CCc2cc(O)c(C(C)(C)C)cc2O1 ; CC1(COc2ccc(CC3SC(=O)NC3=O)cc2)CCc2cc(O)ccc2O1]
or a pharmaceutically acceptable salt thereof.

10. The method of claim 1, wherein said agonist does not have substantial PPAR-α or β/δ agonist activity.

11-12. (canceled)

13. The method of claim 1, wherein the said agonist comprises a compound comprising thiazolidinedione (TZD) domain or a derivative thereof.

14. The method of claim 1, wherein said agonist comprises (RS)-5-(4-[(6-hydroxy-2,5,7,8-tetramethylchroman-2-yl)methoxy]benzyl)thiazolidine-2,4-dione or pharmaceutically acceptable salt thereof.

15. The method of claim 1, wherein the RPE cells are within the eye of a subject, and said agonist is administered to the subject.

16. (canceled)

17. The method of claim 16, wherein said subject is a human of at least 40 years of age or at least 50 years of age.

18-21. (canceled)

22. The method of claim 1, wherein said agonist is administered systemically or locally to the eye by topical application or by ocular injection.

23. The method of claim 1, wherein said agonist is administered locally to the eye by eye drop or by periorbital injection.

24. (canceled)

25. The method of claim 1, wherein said agonist is administered prior to detection of (a) drusen; (b) dry AMD; (c) advanced AMD; (d) advanced AMD and drusen deposits; and/or (e) advanced AMD and significant drusen deposit, in the eye.

26. (canceled)

27. The method of claim 1, wherein the RXR antagonist comprises UVI3003.

28. (canceled)

29. A method for reducing RPE cell death, reducing the size of GA, inhibiting progression of GA, or inhibiting the progression of dry AMD, comprising contacting a RPE cell with a compound, wherein said compound has a structure according to Formula (I), 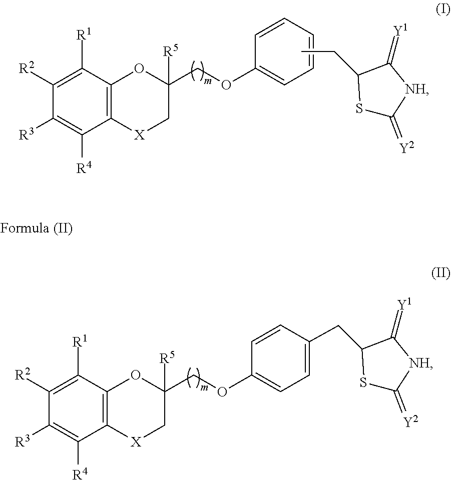
or a pharmaceutically acceptable salt thereof,
wherein:
each of R¹ and R² is independently hydrogen, C₁-C₆ alkyl, or a C₁-C₆ alkoxy group, or R¹ and R² are joined to form a —O(CH₂)ₙO— group;
R³ is hydrogen, —R^{3A}, —OH, —OR^{3A}, —OC(═O)R^{3A}, or —OC(═O)OR^{3A},
R^{3A} is C₁-C₆ alkyl, C₆-C₁₀ aryl, 5- to 6-membered heteroaryl, C₃-C₈ cycloalkyl, or a three- to eight-membered heterocycloalkyl;
R⁴ is a hydrogen atom or a C₁-C₆ alkyl group;
R⁵ is a hydrogen atom or a C₁-C₆ alkyl group;
X is —CH₂—, —C(═O)—, or —CHOR^{X}—;
R^{X} is C₁-C₆ alkyl, C₆-C₁₀ aryl, 5- to 6-membered heteroaryl, C₃-C₈ cycloalkyl, or a three- to eight-membered heterocycloalkyl;
each of Y¹ and Y² is independently O, S, or NH;
m is 1, 2, or 3; and
n is 1, 2, 3, or 4.

30. (canceled)

31. The method of claim 29, wherein:
X is —CH₂—;
R⁵ is hydrogen or unsubstituted C₁-C₆ alkyl;
each of R¹, R², and R⁴ is independently hydrogen or unsubstituted C₁-C₆ alkyl; or
R³ is —OH or —OR^{3A}.

32-34. (canceled)

35. The method of claim 29, wherein said compound is: 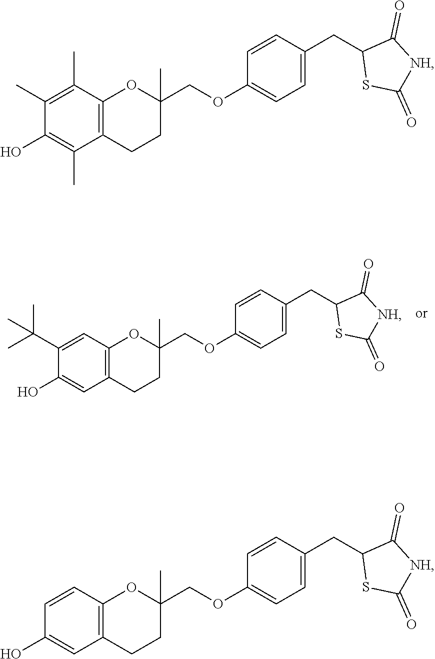 [SMILES: Cc1c(C)c2c(c(C)c1O)CCC(C)(COc1ccc(CC3SC(=O)NC3=O)cc1)O2 ; CC1(COc2ccc(CC3SC(=O)NC3=O)cc2)CCc2cc(O)c(C(C)(C)C)cc2O1 ; "or" ; CC1(COc2ccc(CC3SC(=O)NC3=O)cc2)CCc2cc(O)ccc2O1]
or a pharmaceutically acceptable salt thereof.

36. A method for reducing RPE cell death, reducing the size of GA, inhibiting progression of GA, or inhibiting the progression of dry AMD comprising contacting a RPE cell with a compound, wherein said compound has a structure according to Formula (III), 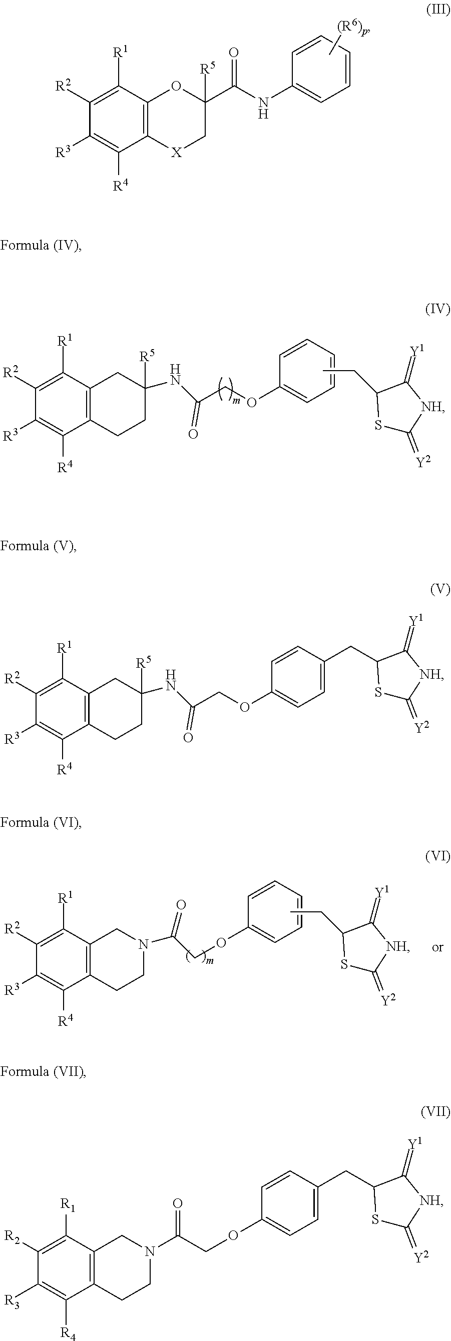
or a pharmaceutically acceptable salt thereof,
wherein:
each of R¹ and R² is independently hydrogen, C₁-C₆ alkyl, or a C₁-C₆ alkoxy group, or R¹ and R² are joined to form a —O(CH₂)ₙO— group;
R³ is hydrogen, —R^{3A}, —OH, —OR^{3A}, —OC(═O)R^{3A}, or —OC(═O)OR^{3A},
R^{3A} is C₁-C₆ alkyl, C₆-C₁₀ aryl, 5- to 6-membered heteroaryl, C₃-C₈ cycloalkyl, or a three- to eight-membered heterocycloalkyl;
R⁴ is a hydrogen atom or a C₁-C₆ alkyl group;
R⁵ is a hydrogen atom or a C₁-C₆ alkyl group;
R⁶, when present, is independently selected from halogen, —CN, —NO₂, C₁-C₆ alkyl, C₆-C₁₀ aryl, 5- to 6-membered heteroaryl, C₃-C₈ cycloalkyl, and a three- to eight-membered heterocycloalkyl;
X is —CH₂—, —C(═O)—, or —CHOR^{X}—;
each R^{X} is independently C₁-C₆ alkyl, C₆-C₁₀ aryl, 5- to 6-membered heteroaryl, C₃-C₈ cycloalkyl, or a three- to eight-membered heterocycloalkyl;
each of Y¹ and Y² is independently O, S, or NH;
m is 1, 2, or 3;
n is 1, 2, 3, or 4; and
p is 0, 1, 2, 3, 4, or 5.

37. The method of claim 36, wherein X is —CH₂—, or R⁵ is hydrogen or unsubstituted C₁-C₆ alkyl.

38-61. (canceled)

62. The method of claim 1, wherein said agonist is selected from the group consisting of: 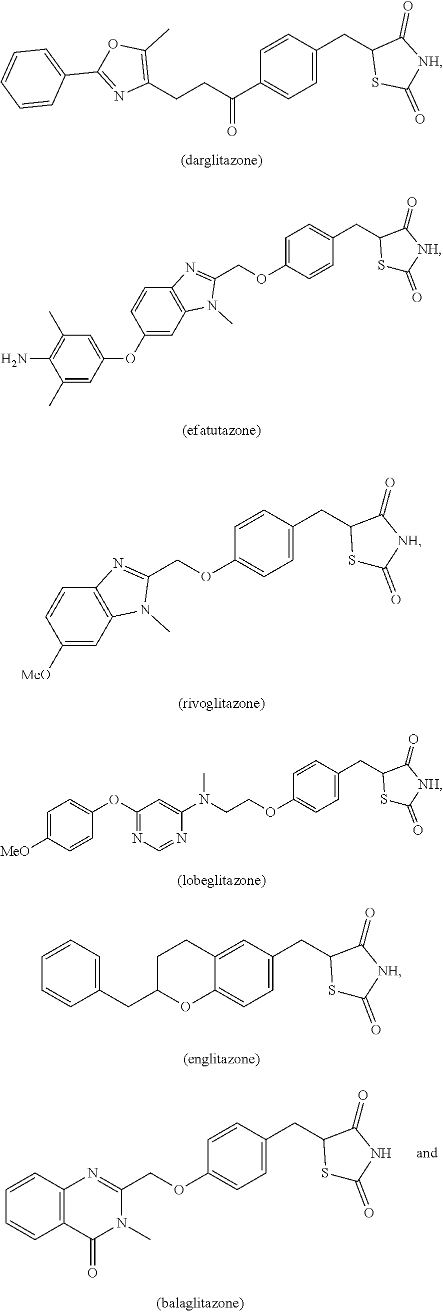 [SMILES: Cc1oc(-c2ccccc2)nc1CCC(=O)c1ccc(CC2SC(=O)NC2=O)cc1 ; Cc1cc(Oc2ccc3nc(COc4ccc(CC5SC(=O)NC5=O)cc4)n(C)c3c2)cc(C)c1N ; COc1ccc2nc(COc3ccc(CC4SC(=O)NC4=O)cc3)n(C)c2c1 ; COc1ccc(Oc2cc(N(C)CCOc3ccc(CC4SC(=O)NC4=O)cc3)ncn2)cc1 ; O=C1NC(=O)C(Cc2ccc3c(c2)CCC(Cc2ccccc2)O3)S1 ; Cn1c(COc2ccc(CC3SC(=O)NC3=O)cc2)nc2ccccc2c1=O] 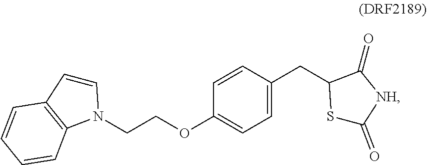 [SMILES: O=C1NC(=O)C(Cc2ccc(OCCn3ccc4ccccc43)cc2)S1]
or a pharmaceutically acceptable salt thereof
